(19) United States
(12) Patent Application Publication
(10) Pub. No.: US 2015/0359909 A1
(43) Pub. Date: Dec. 17, 2015

(54) ORALLY ADMINISTERED BACTERIA AS VEHICLES FOR SYSTEMIC DELIVERY OF AGENTS

(71) Applicant: UNIVERSITY COLLEGE CORK-NATIONAL UNIVERSITY OF IRELAND, CORK, Cork (IE)

(72) Inventors: Gerald O'SULLIVAN, Curaheen (IE); Mark TANGNEY, Glanmire (IE); Douwe VAN SINDEREN, Carrigrohane (IE); Michelle CRONIN, Belgooly (IE)

(21) Appl. No.: 14/799,154

(22) Filed: Jul. 14, 2015

Related U.S. Application Data
(63) Continuation-in-part of application No. 13/384,157, filed on Mar. 22, 2012, now Pat. No. 9,103,831.

(30) Foreign Application Priority Data
Jul. 16, 2009 (EP) 09165716.3

(51) Int. Cl. A61K 49/00; A61K 47/48; A61K 35/74

(52) U.S. Cl. CPC A61K 49/0097; A61K 35/74; A61K 47/48776

(57) ABSTRACT

The current invention relates to the use of a bacterial species in the preparation of a composition adapted for oral administration for the delivery of an agent to a site in the body. The site in the body may be an organ or a tumour site. The bacterial species is a food grade, non-pathogenic, gram-positive bacteria capable of anaerobic growth.

### CROSS-REFERENCE TO RELATED APPLICATION

This application is a continuation-in-part of, and claims priority to, patent application Ser. No. 13/384,157, filed Mar. 22, 2012, entitled ORALLY ADMINISTERED BACTERIA AS VEHICLES FOR SYSTEMIC DELIVERY OF AGENTS, which is a National Stage Application under 35 U.S.C. §371 of International Application No. PCT/EP2010/060363, filed Jul. 16, 2010, entitled ORALLY ADMINISTERED BACTERIA AS VEHICLES FOR SYSTEMIC DELIVERY OF AGENTS, which claims the benefit of Irish Patent Application No. 09165716.3, filed Jul. 16, 2009, the entirety of all of which is incorporated herein by reference.

### STATEMENT REGARDING FEDERALLY SPONSORED RESEARCH OR DEVELOPMENT

n/a

### TECHNICAL FIELD

The present invention relates to bacterial translocation. More specifically, the present invention relates to methods of using bacteria, in particular orally administered bacteria, as a vehicle for delivery of agents to a site in the body.

### BACKGROUND

The current challenges facing oncology practitioners and researchers include the development of specific treatments and efficient tumour detection methods. Genetically modified, pathogenic and non-pathogenic bacteria have begun to emerge as potential biological agents with natural tumour specificity. Several bacterial species or genera (e.g. Escherichia coli, Bifidobacterium, attenuated Salmonella typhimurium, Clostridium, Vibrio cholera, Listeria monocytogenes) have been demonstrated to localise to and replicate in tumour tissue when intravenously administered in rodent models. Achieved through anaerobic bacterial targeting, specific localisation of species, such as members of the Bifidobacterium genus, in hypoxic regions of tumours after intravenous administration has been reported.

The term bacterial translocation refers to trafficking of viable bacteria from the Gastro-Intestinal Tract (GIT) to extraintestinal sites. This phenomenon is well studied in bacterial sepsis linked with various conditions^{14,15}. The three primary mechanisms promoting bacterial translocation in animal models are identified as: (a) disruption of the ecologic GI equilibrium to allow intestinal bacterial overgrowth, (b) increased permeability of the intestinal mucosal barrier, and (c) deficiencies in host immune defenses. These mechanisms can act in concert to synergistically promote the systemic spread of indigenous translocating bacteria to cause lethal sepsis. In animal models in which the intestinal barrier is not physically damaged, pathogenic bacteria may translocate by an intracellular route through the epithelial cells lining the intestines and then travel via the lymph to the mesenteric lymph node (MLN). In animal models exhibiting damage to the mucosal epithelium, indigenous bacteria translocate intercellularly between the epithelial cells to directly access the blood. Indigenous gastro-intestinal (GI) bacteria have been cultured directly from the MLN of various types of patients. Thus, evidence is accumulating that translocation of indigenous bacteria from the GI tract is an important early step in the pathogenesis of opportunistic infections originating from the GI tract. Sampling from humans has indicated that translocation may be a phenomenon that occurs in healthy individuals and may be a normal physiologic event without deleterious consequences⁴⁸.

Bifidobacteria are a native, harmless resident of the human gut, and certain bifidobacterial strains have been shown to have health-promoting or probiotic benefits. A number of bifidobacterial strains that harbour plasmids expressing therapeutic agents, such as endostatin or prodrug activating enzymes, have been shown to induce regression in rodent tumour models when administered intravenously³³. Live imaging of mice has shown intravenously administered lux tagged invasive pathogenic bacteria replicating locally in tumours.

To date, bacterial localisation to tumours has been described only with intravenous administration. Fu et at previously described use of B. longum to deliver a therapeutic peptide to the GIT³³. B. longum expressing endostatin was administered orally to athymic mice, and the authors reported that subsequent gut absorption of the therapeutic peptide resulted in slowing in the growth of subcutaneous (s.c.) liver tumours. Translocation of bacteria from the gut to tumours was not reported. Several studies have investigated the ability of bifidobacterial colonisation of the GIT to inhibit translocation of pathogens¹⁶. A study from 1985 demonstrated B. longum colonisation of organs post feeding⁴⁷. The authors reported very low bacterial counts in the kidney and liver however. In addition the authors did not report bacterial translocation to tumour cells.

Despite the teachings of the prior art, the current inventors have surprisingly demonstrated translocation of a non-pathogenic species of bacteria following oral administration, utilising a lux luminescence-based tagging system in Bifidobacteria breve in mice, with subsequent growth specifically in tumours. B. breve UCC2003 carrying the plasmid pLuxMC3¹⁸ was orally administered to athymic MF1 nu/nu mice bearing subcutaneous (s.c.) B16-F10 murine melanoma tumours (FIG. 1a). By transfection with plasmids that are suitable for bacterial replication expression of heterologous genes, such bacteria can home to tumours, replicate within them and locally express therapeutic proteins. It would not have previously been expected by those skilled in the art that non-pathogenic viable species of bacteria would translocate to tumours in hosts.

The current invention has the added advantage in that it necessitates oral administration, which is relatively painless and offers greater flexibility for use in a range of clinical situations and is of particular benefit for pediatric patients. The administration of therapeutic agents intravenously, as described previously, is painful and causes patient discomfort.

It is an object of the current invention to provide a method of using bacterial species as vehicles to deliver agents. The agents and/or the bacterial species are delivered to a site in the body, which may be an organ or a tumour or the bloodstream. A further object of the current invention is to provide a method of tumour detection, a method of tumour imaging, a method of monitoring the effectiveness of tumour treatment, a method of tumour staging, a method of detecting tumour metastasis, and/or in a method of tumour or cancer treatment. It is a further object of the invention to provide a therapeutic agent for the treatment of disease.

### SUMMARY

According to the first aspect of the current invention there is provided a method of delivering an agent to an extra-intestinal site in the body comprising orally administering a composition to the subject, the composition comprising at least one bacterial species adapted for oral administration, the bacterial species either being linked to the agent or being capable of expressing the agent, wherein the bacterial species targets a site in the body and the agent is delivered within or adjacent to the site in the body. In a preferred embodiment, the bacterial species is deliverable to a site in the body and is capable of expressing, releasing or producing an agent at the site. In other words, the bacterial species is capable of passing from the intestine into the bloodstream or into an organ of the body.

In one embodiment the agent and/or bacterial species is deliverable to an organ. The organ may be any organ of the body. The site in the body may be an organ, or the bloodstream or a tumour site. In a preferred embodiment the bacterial species is viable. The agent may be encoded on a vector capable of expressing at least one gene, the vector being transformed into the bacterial species. In an alternative embodiment the agent may be linked to the bacterial species. The agent may be linked, bonded, fixed or chemically coupled or attached to the bacterial species or the bacterial membrane or attached/linked by any other means known to the skilled person.

In a still further embodiment the bacterial species is adapted for oral administration for the delivery of an agent to an organ, a tumour, or the bloodstream wherein the bacterial species is deliverable to those sites.

In a preferred embodiment of the method the agent and/or bacterial species is deliverable to a tumour site.

In this preferred embodiment the composition or bacterial species may be adapted for or is for use in a method of tumour detection, a method of tumour imaging, a method of monitoring the effectiveness of tumour treatment, a method of tumour staging, a method of detecting tumour metastasis, and/or in a method of tumour or cancer treatment. The method of tumour detection and treatment may occur simultaneously. The agent may be an imaging agent, such as a fluorescent protein (e.g. RFP, GFP) a substrate-converting enzyme (e.g., Luciferase, bacterial lux operon, Alkaline Phosphatase, B-Galactosidase), or image contrast-enhancing agent. Alternatively the agent may be a tumour suppressing agent, such as an anti-angiogenic agent (e.g. endostatin, angiostatin, IL12, IL23, IL32), an immune cell regulating agent (cytokine or other), a secreted peptide, a tumour associated antigen, an antibody, a pro-apoptotic peptide (e.g. IL23), or a cell signalling interfering peptide (e.g. IL 32).

The gene may encode an agent selected from the group comprising an imaging agent, such as a fluorescent protein (e.g. RFP, GFP) a substrate converting enzyme (e.g., Luciferase, bacterial lux operon, Alkaline Phosphatase, B-Galactosidase), an image contrast enhancing agent, a tumour suppressing agent, such as an anti-angiogenic agent (e.g. endostatin, angiostatin, IL12, IL23, IL32), an immune cell regulating agent (cytokine or other), a secreted peptide, a tumour associated antigen, an antibody, a pro-apoptotic peptide (e.g. IL23), or a cell signalling interfering peptide (e.g. IL 32).

In all embodiments, the bacterial species may be any non-pathogenic bacterium. The bacterial species may be viable. The non-pathogenic bacterium may be an E. coli, Bifidobacterium, Lactobacillus or a Lactococcus species.

The bacterium may be an E. coli species selected from E. coli Nissle 1917 or E. coli K12-related strain such as MG1655.

The bacterium may be a Bifidobacterium selected from the group comprising B. breve, B. adolescentis, B. angulatum, B. bifidum, B. parvolulorum, B. catenulatum, B. denticolens, B. dentium, B. gallicum, B. infantis, B. liberorum, B. lactenti, B. inopinatum, B. longum, B. pseudocatenulatum, B. lactis, B. animalis, B. minimum, B. subtile, B. thermacidophilum, B. asteroids, B. boum, B. choerinum, B. coryneforme, B. cuniculi, B. gallinarum, B. indicum, B. magnum, B. merycicum, B. pseudolongum, B. pullorum, B. ruminantium, B. saeculare, B. suis, B. thermophilum, B. ruminale, B. asteroids, B. boum, B. gallinarum, B. minimum.

The bacterium may be a Lactobacillus species selected from the group comprising L. acetotolerans, L. acidifarinae, L. acidipiscis, L. acidophilus, L. agilis, L. algidus, L. alimentarius, L. amylolyticus, L. amylophilus, L. amylotrophicus, L. amylovorus, L. animalis, L. antri, L. apodemi, L. aviarius, L. bifermentans, L. brevis, L. buchneri, L. camelliae, L. casei, L. catenaformis, L. ceti, L. coleohominis, L. collinoides, L. composti, L. concavus, L. coryniformis, L. crispatus, L. crustorum, L. curvatus, L. delbrueckii subsp. delbrueckii, L. delbrueckii subsp. bulgaricus, L. delbrueckii subsp. lactis, L. dextrinicus, L. diolivorans, L. equi, L. equigenerosi, L. farraginis, L. farciminis, L. fermentum, L. fornicalis, L. fructivorans, L. frumenti, L. fuchuensis, L. gallinarum, L. gasseri, L. gastricus, L. ghanensis, L. graminis, L. hammesii, L. hamsteri, L. harbinensis, L. hayakitensis, L. helveticus, L. hilgardii, L. homohiochii, L. iners, L. ingluviei, L. intestinalis, L. jensenii, L. johnsonii, L. kalixensis, L. kefiranofaciens, L. kefiri, L. kimchii, L. kitasatonis, L. kunkeei, L. leichmannii, L. lindneri, L. malefermentans, L. mali, L. manihotivorans, L. mindensis, L. mucosae, L. murinus, L. nagelii, L. namurensis, L. nantensis, L. oligofermentans, L. oris, L. panis, L. pantheris, L. parabrevis, L. parabuchneri, L. paracollinoides, L. parafarraginis, L. parakefiri, L. paralimentarius, L. paraplantarum, L. pentosus, L. perolens, L. plantarum, L. pontis, L. psittaci, L. rennini, L. reuteri, L. rhamnosus, L. rimae, L. rogosae, L. rossiae, L. ruminis, L. saerimneri, L. sakei, L. salivarius, L. sanfranciscensis, L. satsumensis, L. secaliphilus, L. sharpeae, L. siliginis, L. spicheri, L. suebicus, L. thailandensis, L. ultunensis, L. vaccinostercus, L. vaginalis, L. versmoldensis, L. vini, L. vitulinus, L. zeae, L. zymae.

The bacterium may be a Lactococcus species selected from the group comprising L. garvieae, L. lactis, L. piscium, L. plantarum, L. raffinolactis.

In another aspect, the current invention provides a method of delivering an agent to a tumour site comprising the use of a bacterial species adapted for oral delivery. The invention also provides a method of delivering an agent to the bloodstream comprising the use of a bacterial species adapted for oral delivery, which may enable body-wide circulation of bacteria. In such methods the bacterial species is deliverable to the tumour or the bloodstream. The bacterial species may be any non-pathogenic, bacterium capable of anaerobic growth.

In another embodiment the current invention provides a method of delivering an agent to a site in the body, such as an organ or a tumour site or the bloodstream, comprising the use of a bacterial species adapted for oral delivery. The bacterial species may be deliverable to the site in the body. The method comprising orally administering a composition to a subject, the composition comprising a bacterial species containing a vector capable of expressing at least one exogenous gene, wherein the bacterial species targets a site in the body and the gene is expressed within or adjacent to the site in the body. The bacterial species may be any food grade, non-pathogenic, gram-positive bacteria capable of anaerobic growth.

The invention further provides a method of tumour detection or tumour imaging comprising orally administering a composition comprising, a bacterial species containing a vector capable of expressing at least one exogenous gene, wherein the bacterial species targets the tumour environment and the gene is expressed within or adjacent to the targeted tumour. The method further comprises external imaging and/or detecting/measuring the expression of the gene, to determine the targeted sites in the body to allow detection/imaging of said tumours in the body.

The invention further provides a method of monitoring the effectiveness of tumour treatment, tumour staging or detecting tumour metastasis comprising orally administering a composition comprising, a bacterial species containing a vector capable of expressing at least one exogenous gene, wherein the bacterial species targets the tumour environment and the gene is expressed within the targeted tumour. The method further comprises external imaging and/or detecting/measuring the expression of the gene, to determine the targeted sites in the body to allow the monitoring the effectiveness of tumour treatment, tumour staging or detecting tumour metastasis of said tumours in the body.

The gene may encode an agent selected from the group comprising an imaging agent, such as a fluorescent protein (e.g. RFP, GFP) a substrate converting enzyme (e.g., Luciferase, bacterial lux operon, Alkaline Phosphatase, B-Galactosidase), an image contrast enhancing agent, a tumour suppressing agent, such as an anti-angiogenic agent (e.g. endostatin, angiostatin, IL12, IL23, IL32), an immune cell regulating agent (cytokine or other), a secreted peptide, a tumour associated antigen, an antibody, a pro-apoptotic peptide (e.g. IL23), or a cell signalling interfering peptide (e.g. IL 32).

It is a further aspect of the invention to provide a bacterial species adapted for oral administration in the preparation of a composition for the delivery of an agent to a site in the body wherein the composition is adapted for use as a therapeutic agent for the treatment of disease and/or a diagnostic agent for the detection of disease. The site may be an organ, a tumour or the bloodstream.

The term “vector” used herein refers to any DNA material capable of transferring genetic material into a host organism. The vector may be linear or circular in topology and includes but is not limited to plasmids, food grade plasmids, DNA bacteriophages or DNA viruses.

### BRIEF DESCRIPTION OF THE DRAWINGS

The current invention will now be described with reference to the following examples and figures. It is to be understood that the following detailed description and accompanying figures, are exemplary and explanatory only and are intended to provide a further explanation of the present invention, as claimed and not to limit the scope of the invention in any way.

FIG. 1: (a) Protocol for animal trials. Subcutaneous tumours were induced in mice, and bifidobacteria administered upon tumour development. For oral administration, each animal received 10⁹ B. breve UCC2003 on three consecutive days. For intravenous (i.v.) administration, each animal received 10⁴ cells injected directly into the lateral tail vein. Mice were imaged (IVIS) immediately prior to culling with subsequent recovery of viable bacteria (cfu) from tissues (arrows);

FIG. 1: Trafficking of UCC2003 to subcutaneous tumours. B. breve UCC2003 following either oral (b) or intravenous (c) oral delivery, displaying recovery from s.c. tumour tissue and other organs (bars, y axis) and lux expression in vivo in live MF1nu/nu mice (squares, z axis and images). Increase in bacterial numbers and plasmid gene expression specifically in tumours was observed over time. Low levels of B. breve colony forming units were detected in all organs examined from all treated animals (≦100 cfu/organ; no detectable luminescence);

FIG. 1: (d) Comparative recovery of UCC2003 from subcutaneous tumours following either oral or intravenous delivery. No significant differences between oral and intravenous administration of UCC2003 were observed in bacterial recovery (y axis) or luminescence (z axis) from s.c. tumours. UCC2003 cfu counts were similar on day 7 (p=0.360), with i.v. counts higher on day 11 (p=0.038) and oral counts higher on day 14 (p=0.021);

FIG. 2: Bifidobacterial translocation in immunocompetent mice. UCC2003 recovery following either (a) oral or (b) intravenous delivery to athymic and immunocompetent mice. Bacterial recovery (y axis) and luminescence (z axis) from s.c. tumour tissue and various organs are represented. UCC2003 recovery from organs was comparable between both species and delivery mechanisms. Detectable luminescence and significantly higher bacterial recovery were observed in tumours of all mice relative to other tissues;

FIG. 3: Influence of tumour on systemic bifidobacterial spread. Cfu levels in blood and organs were compared between tumour bearing and tumour-free mice orally administered UCC2003. (a) The presence of tumour did not significantly alter levels of cfu recovery from liver, kidney or blood (p>0.240). (b) Bacterial colonisation of B16 pulmonary metastases. Significantly increasing bacterial load was observed over time in lungs of tumour bearing animals (*Day 7 p=0.045, day 11 p=0.030, day 14 p=0.006) indicating bifidobacterial colonisation and replication in metastatic nodules;

FIG. 4: Correlation between bacterial counts and luminescence. A linear relationship between luminescence and bacterial counts was observed up to 10⁵ cfu. At higher bacterial load, luminescence underestimated. Since the activity of BioLuminescence Imaging systems are dependent on both ATP and oxygen availability^{57,58}, lower luminescence (than expected based on bacterial cell counts) may be the result of decreased metabolic activity and/or reduced oxygen availability at higher bacterial concentrations and/or larger more hypoxic tumours. ATP, adensine triphosphate; cfu, colony forming units;

FIG. 5: Systemic localisation of UCC2003 administered orally or intravenously. Bacterial counts in organs were compared between oral and i.v. administered tumour free athymic or immunocompetent C57 mice. Organ colonisation was similar in all cases;

FIG. 6: Bifidobacterial translocation in immunocompetent mice. (a) B. breve colonisation of tumours post oral or i.v. administration to immunocompetent animals. B. breve UCC2003 recovery (y axis) and luminescence (z axis) from B16-F10 tumours following either oral or intravenous delivery to immunocompetent C57 mice. Bacterial levels in tumours were comparable whether UCC2003 was orally or i.v administered. In all cases, detectable luminescence and significantly higher bacterial recovery (p<0.003) were observed in tumours relative to other tissues. Sample IVIS images from day 11 are shown. (b) Comparison between B. breve levels in athymic and immune competent mice. Following oral administration of UCC2003, there were no significant differences (p>0.107) observed between cfu levels in organs from C57 mice (bars) and athymic mice (red dots). Tumour cfu levels were significantly higher in athymic mice, with differences of up to 3-log fold by day 14;

FIG. 7: Cellular and humoural immune responses to UCC2003. (a) T cell levels in bifidobacteria-colonised tumours. Flow Cytometry analysis was used to quantify CD3-positive T cells in s.c. tumours from C57 mice orally administered UCC2003 or PBS. A 25.8 (+/−5.8) % increase in total T cell numbers was observed in UCC2003 colonised tumours 11 days post feeding, compared with unfed mice (p=0.06, n=3). (b) Humoural responses. IgG antibody specific for UCC2003 was measured by ELISA from serum taken from immunocompetent C57 mice 14 days post administration (n=3). Results indicated that antibodies were raised against UCC2003. There was no significant difference between anti-UCC2003 IgG antibody levels in serum from IV and fed mice (p=0.359);

FIG. 8: (a) and (b) Recovery of various bacterial genera from murine tissues. Low levels (5-50 cfu) of indigenous commensal bacteria (Lactobacilli, Enterobacteriaceae, Staphlococci, Micrococci and Bacteroides) were recovered from all organs examined, but not blood, in both immunocompetent and athymic mice. No differences in non-UCC2003 bacterial levels were observed between UCC2003-fed and control (PBS-fed) mice (p>0.114), except for decreases in Gram-negative bacteria in lung, liver and kidney of C57 mice fed UCC2003 (p<0.044), indicating inhibition of certain species by UCC2003;

FIG. 9: Trafficking of orally-administered E. coli Nissle 1917 and Bifidobacterium infantis 35624 to tumours in mice. Probiotic bacteria E. coli Nissle 1917/p16Slux (lux-expressing; Em-resistant) or Bifidobacterium infantis 35624/pAM5 (Tet-resistant), were orally administered to mice bearing subcutaneous RENCA renal tumours. Tumour localisation was observed in both cases, mirroring the observations with B. breve UCC2003;

FIG. 10: Comparison of orally-administered bacterial species in tumours in mice. MF1-nu/nu mice bearing subcutaneous RENCA tumours were gavaged ×3 days with 100 μl@ 10e9 each of either B. breve UCC2003luxABCDE (n=5); B. infantis 35624pAM5 (n=6); E. coli Nissle p16Slux (n=6) or PBS (n=3). Mice were supplied streptomycin at 5 g/L added in drinking water for three days before bacterial administration and throughout the experiment, to deplete the native flora; and

FIG. 11: Detection of probiotic bacteria fed to patient tumours. PCR specific for Bifidobacterium infantis or E. coli Nissle was performed with DNA extracted from cancer patient samples. Fresh tumour and normal tissue samples were obtained from patients undergoing resection of lung or kidney tumours. Patients eat B. infantis (Align), E. coli Nissle 1917 (Mutaflor) or no probiotic daily for ≧3 weeks immediately prior to surgery. Strain-specific PCR indicated the presence of this strain only in tissue of patients fed the respective bacterium, both in lung and kidney cancer patients, indicating translocation of these probiotics from the GIT.

### DETAILED DESCRIPTION

### Materials and Methods

### Cell Culture

B. breve UCC2003 or Bifidobacterium 35624 (UCC Culture Collection) were routinely grown at 37° C. in reinforced clostridial medium (RCM) (Oxoid, Basingstoke, United Kingdom). For bioluminescence assays MRS medium (Oxoid), supplemented with 0.05% (w/v) cysteine-HCl was used. Anaerobic conditions were maintained using an anaerobic chamber [Mac500, Don Whitley Scientific, West Yorkshire, UK (atmosphere 10% H₂, 10% CO₂, 80% N2)]. B. breve UCC2003/pLuxMC3¹⁸, expressing the luxABCDE operon from the Pₕₑₗₚ promoter⁵², and Bifidobacterium 35624/pAM5 were cultured in the presence of 4 μg/ml chloramphenicol (Cm) or tetracycline (Tet), respectively. To facilitate specific recovery of bifidobacteria from tissue samples, 50 mg mupirocin (Oxoid)/litre was included, as previously described⁵³. E. coli Nissle 1917 engineered with the plasmid p16Slux which contains the constitutive P_{HELP}luxABCDE operon⁶ was routinely grown aerobically at 37 C in LB medium (Sigma, Steinheim, Germany) supplemented with 300 mg/ml erythromycin (Em). B16-F10 and RENCA (ATCC) were maintained in DMEM (GIBCO, Invitrogen Corp., Paisley, Scotland) supplemented with 10% FBS. Cell densities were determined by visual count using a haemocytometer and viable cell counts were conducted using Trypan Blue Dye Exclusion (Gibco).

### Animals and Tumour Induction

All in vivo experiments were approved by the ethics committee of University College Cork. For routine tumour induction, 1×10⁶ B16-F10 tumour cells, suspended in 200 μl of serum-free DMEM (Sigma), were injected subcutaneously (s.c.) into the flank of 6-8 week old female C57 or MF1-nu/nu mice (Harlan, UK). Tumours were monitored mostly by alternate day measurements in two dimensions using a verniers calliper. Tumour volume was calculated according to the formula V¼ab2P/6, where a is the longest diameter of the tumour and b is the longest diameter perpendicular to diameter a.

### Bacterial Administration

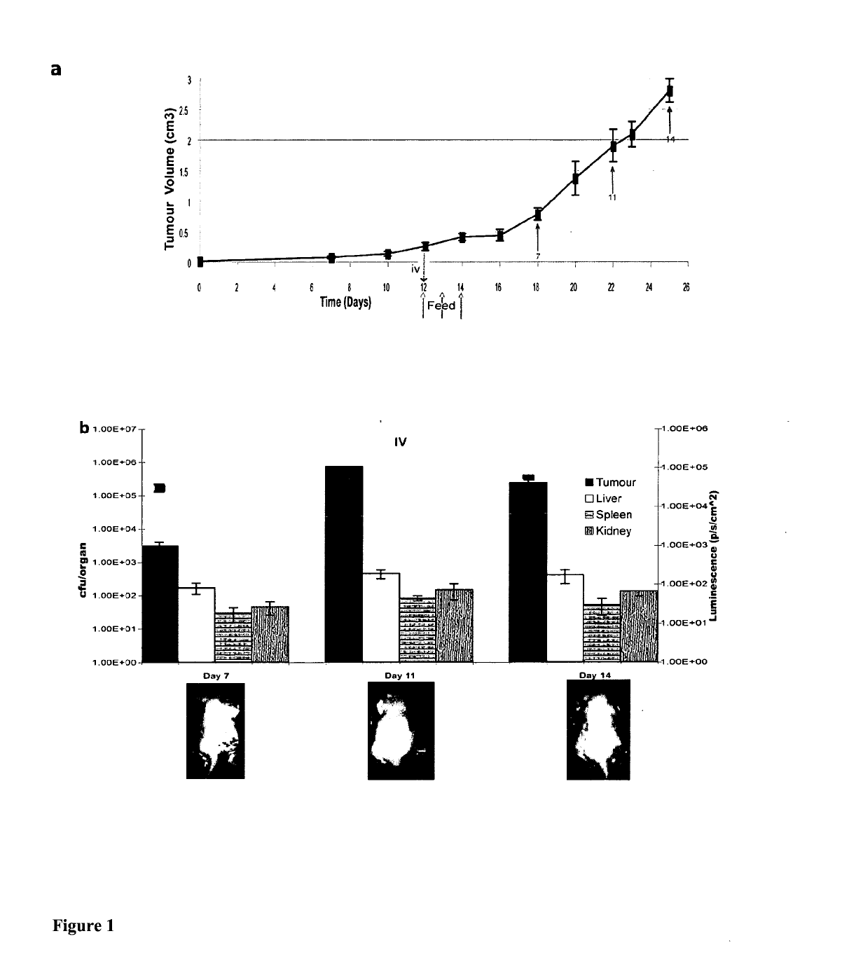

The inocula were prepared by growing B. breve UCC2003 containing pLuxMC3 anaerobically overnight at 37° C. in 100 ml of MRS broth containing 4 μg/ml Cm. Cultures were harvested by centrifugation (6,000×g for 5 min), washed with PBS supplemented with 0.05% cysteine HCl (Sigma), and resuspended in a one-tenth volume of PBS. When tumours reached approximately 100 mm³ in volume, mice were randomly divided into experimental groups (n=6) and administered bifidobacteria or an equal volume of PBS as control. For oral administration, 10⁹ cells was administered in 20 μl per mouse on three consecutive days using a micropipette tip placed immediately behind the incisors. For intravenous administration, each animal received 10⁴ cells in 100 μl injected directly into the lateral tail vein, which we found to be the maximum injectable dose with this strain (FIG. 1). The viable count of each inoculum was determined by retrospective plating on Reinforced Clostridial Agar (RCA) containing 4 μg/ml Cm.

### Whole Body Imaging

On day 7, 11 and 14-post first inoculation, animals were anesthetised by intraperitoneal administration of 200 mg xylazine and 2 mg ketamine, and whole-body image analysis was performed in the Xenogen IVIS 100 system for 5 minutes at high sensitivity.

Bacterial Recovery from Mice

Following imaging, a subset of animals from each group were euthanised by cervical dislocation. Cardiac puncture was immediately performed to obtain blood, and subsequently individual tumours as well as lungs, liver, spleen and kidneys were asceptically removed and examined for bioluminescence. Ex vivo IVIS imaging detected no luminescence in organs. Following imaging, each tissue was homogenized by fine mincing by scalpel followed by pushing through a 20 μm pore nylon filter (Falcon, Becton Dickinson (BD), Oxford, England) in sterile PBS supplemented with 0.05% cysteine-HCl. Serial dilutions were plated in duplicate on RCA agar containing 4 μg/ml Cm and mupirocin. Resulting colonies were used to calculate the number of UCC2003 cells per tissue sample. To confirm that cfu recovered were B. breve/pLuxMC3, random isolates were spot inoculated onto RCA only or RCA containing Cm 4 μg/ml. Specific identification of UCC2003 pLuxMC3 was confirmed by PCR using primer pairs targeting both the intergenic spacer region and the unique apuB gene on the chromosome of UCC2003 (5′-GGTGTGAAAGTCCATCGCT-3′ (SEQ ID NO: 1) and 5′-GTCTGCCAAGGCATCCACCA-3′ (SEQ ID NO: 2))¹⁹.

To facilitate the recovery of other bacteria potentially translocating from the GIT, total tissue homogenates were serially diluted and plated on MRS agar (Oxoid) to select for aerobic Gram-positive bacteria and on McConkey agar (Oxoid) to select for Gram-negative bacteria. Plates were incubated for 24-48 h and cfu enumerated. Morphology of differing colony types was assessed and the colonies re-streaked. Following catalase and Gram reaction tests, five colony types were subjected to API analysis (BioMerieux) to determine species.

### DNA Extraction and Quantitative PCR

Tumours (average volume 1 cm³) were excised from MF1 nu/nu mice 11 days post oral administration of UCC2003/pLuxMC3. Each tumour was aseptically dissected to separate the periphery from the central region. Necrosis was evident in the centre of tumours of this size. Total DNA was extracted from each segment using the Sigma GenElute DNA extraction kit with the following modifications; DNA samples were flash frozen in liquid nitrogen and homogenised; digestion of the tissue was in lysis buffer containing 40 mg/ml proteinase K and incubation was increased to overnight at 55° C. As a UCC2003-free control, DNA was also extracted from the tumour of a naïve mouse. The concentration of DNA in all samples was determined using the Nanodrop system (ND-1000 Spectrophotometer, Labtech Int, East Sussex, UK), and equal concentrations of DNA added to each PCR reaction. The concentration of the mammalian cell housekeeping gene Glyceraldehyde-3-phosphate dehydrogenase (GAPDH) was also determined for all samples to ensure parity between PCR template DNA samples. Quantitative PCR (qPCR) was performed using the LightCycler system (Roche Diagnostics, U.K.). A 20 μl reaction contained 0.5 μM of each sense and anti-sense primer, 4 mM MgCl₂, 2 IA LightCycler FastStart DNA Master SYBR Green and 100 ng of template DNA. PCR conditions were 95° C. for 10 min; 95° C. for 10 s, 56° C. for 5 s for luxA primers (59° C. for GAPDH primers), 72° C. for 5 s. Melting curve: 95° C. for 0 s, 66° C. for 15 s, 99° C. for Os; Cooling step: 40° C. for 30 s. The concentration of pLuxMC3 in each sample was determined using a standard curve generated with pLuxMC3 plasmid DNA, and corresponding plasmid copy number calculated using the formula (Mass DNA/ug=n by plasmid/3×10⁹ bp). pLuxMC3 has an average copy number/cell of 3±0.18 in UCC2003, and resulting plasmid copy numbers were divided by this figure to calculate bacterial cell numbers/μg DNA.

### Flow Cytometry Analysis

Mouse tumours were excised and finely minced using a scalpel. Tissue was subsequently chemically dissociated in DMEM containing Collagenase I (300 U/ml, Sigma Aldrich) and DNase (0.01%, Sigma Aldrich) for 40 min at 37° C. and then applied to a cell strainer (70 μm mesh size, Becton Dickinson, UK). Red blood cells were lysed using ACK erythrocyte lysis buffer (distilled H2O, 0.15 M NH4Cl, 10 mM KHC03, 0.1 mM Na2EDTA) and cell counts determined using a NucleoCounter (Chemometec, Denmark). Cells at a concentration of 1×10⁶ cells per 100 μl were washed in PBS and fixed using 70% ethanol. FACS analysis was performed using anti-mouse CD3e-PE-Cy5 (145-2C11), anti-mouse Fc block (CD16/32) and relevant isotype control as per manufacturer instructions (eBioscience, USA). FACS staining buffer (PBS, 2% FBS, 1% BSA and 0.05% sodium azide) was used as wash and staining buffer. Analysis was performed on a FACSDiva (BD, England) and analysed using BD FACSDiva software 6.0 (BD, England).

### Antibody Detection

Antibody was measured by an indirect-Enzyme Linked ImmunoSorbent Assay (ELISA), as described in Yasui et al.⁵⁶ with the following modifications. B. breve UCC2003 cells (1×10⁸ bacteria/well) were coated on the wells of a 96-well ELISA plate using carbonate buffer pH 9.6 and incubated at room temperature (RT) overnight. Blood samples were collected at necropsy by cardiac puncture from female C57 BL/6 mice administered UCC2003 either orally (n=3) or intravenously (n=3), or untreated mice (n=3), as previously described. To separate plasma, the samples were centrifuged for 10 minutes at 4000×g within 30 minutes of collection. Plates were blocked for 1 hour at 37° C. by the addition of 10% rabbit serum diluted in PBS containing 0.05% NaN3 (PBSN). After washing three times with PBSN, 50 μl of mouse serum samples (1:1, 1:10, 1:100 dilutions) in blocking buffer (0.05% Tween 20 and 0.25% BSA in PBSN) were added to the plates and incubated overnight at RT. All samples were assayed in triplicate. The plates were then blocked again by 1 hour incubation at 37° C. in 10% sheep serum, and further incubated with goat anti-mouse IgG conjugated to alkaline phosphatase (Sigma) for 5 hours at RT. After incubation with the substrate pNPP, qualitative hydrolysis of NPP was detected using a microtiter plate reader (Vmax, Molecular Devices) with a 405-nm filter. Antibody levels are expressed as the means and standard error of the values for three mice per route.

### Cytokine Analysis

MF1nu/nu mice bearing B16 tumours were sacrificed by cervical dislocation at 11 days post-feeding with B. breve UCC2003 pLuxMC3 or PBS. The blood samples were obtained using cardiac puncture, serum separated by centrifugation and used immediately for the cytokine assay. The entire GIT was also extracted, frozen and then homogenised on ice in tissue lysis buffer containing a denaturing detergent (<0.1% SDS) and reducing agent (<1 mM DTT) as well as 1% BSA. The MSD Murine Cytokine 7-plex ultrasensitive panel (Meso Scale Discovery, Gaithersburg, Md., USA) was run as per kit recommendations. This plate analyses murine Interferon-gamma (IFN-γ), Interleukin-1beta (IL-1b), Interleukin-6 (IL-6), Interleukin-10 (IL-10), Interleukin 12 (IL-12p70), Tumor necrosis factor (TNF-α) and the mouse keratinocyte-derived chemokine (mKC), a functional homolog of human interleukin-8.

### Statistical Analysis

Two-tailed Student's t-tests were employed to investigate statistical differences. Microsoft Excel 12 (Microsoft) was used to manage and analyze data. Statistical significance was defined at the standard 5% level.

Patient Study: Trafficking of Orally-Administered E. coli Nissle 1917 and B. infantis 35624 to Patient Tumours

Subjects:

Human subjects were patients with primary renal or lung tumours, scheduled for resection.

Bacterial Feeding Prior to Surgery:

The bacterial preparations used are established, commercially available capsule format preparations—E. coli Nissle 1917 as Mutaflor (Medical Futures Inc. Canada); B. infantis 35624 as Align (Procter & Gamble, USA). Selected, consented patients eat B. infantis (Align), E. coli Nissle 1917 (Mutaflor) or no probiotic daily for a period of ≧3 weeks prior to surgical resection.

Tissue Samples:

At the time of surgery, fresh tissue samples were collected asceptically and flash frozen.

Sample Analysis:

Total DNA was isolated from frozen samples. PCR primers specific for the target strains were utilised to determine the presence of the specific bacterium in patient malignant and/or non-malignant tissue (kidney or lung).

DNA Extraction

DNA extraction from human cancer patient tissue samples was performed using the Gen Elute Mammalian Genomic DNA Miniprep Kit using the standard protocol for tissue samples with the following modifications. The Proteinase K incubation time was increased to overnight (˜16 h) and the amount of Proteinase K added to each samples was increased to 30 μL. Following completion of the protocol samples were equalized to a volume of 50 μL through use of a vacuum centrifuge to reduce the volume.

PCR Amplification and Gel Electrophoresis

A nested PCR was performed using 1×DreamTaq Green PCR Master Mix (Thermo) and primers as below, (10 μM concentration) in 25 μL reactions. The first PCR utilized 2 μL of the extracted tissue DNA as template while the second PCR reaction used 3 μL of the first PCR product as template. Thermocycling conditions for both PCR steps were as follows: initial denaturation 95° C. for 5 min, followed by 35 cycles of 95° C. for 45 sec, 55° C. for 60 sec, and 72° C. for 60 sec, and a final elongation at 72° C. for 7 min. PCR product sizes were confirmed by running 7 μL of PCR product on a 1.5% agarose gel at 130V for 20-30 min. Primers: E. coli: Rounds 1 and 2—E. coli NIssle 1917=Muta9F (5′-GCG AGG TAA CCT CGA ACA TG-3′) (SEQ ID NO: 3)/Muta10R (5′-CGG CGT ATC GAT AAT TCA CG-3′) (SEQ ID NO: 4)—targeting the pMUT2 plasmid⁵. Bifidobacterium: Round 1-PbiF1 (CCG GAA TAG CTC C) (SEQ ID NO: 5)/PbiR2 (GAC CAT GCA CCA CCT GTG AA) (SEQ ID NO: 6) Round 2—BiLON1-F (TTC CAG TTG ATC GCA TGG TC) (SEQ ID NO: 7)/Pb1LonR4 (CGT ATC TCT ACG ACC) (SEQ ID NO: 8).

### Results

### In Vivo Imaging of Bacteria in Tumours

The inventors administered B. breve UCC2003 carrying the plasmid pLuxMC3, which expresses the luminescent bacterial luxABCDE operon under the control of the bacterial Pₕₑₗₚ promoter, by tail vein injection to athymic MF1 nu/nu mice bearing subcutaneous (s.c.) B16-F10 murine melanoma tumours (FIG. 1).

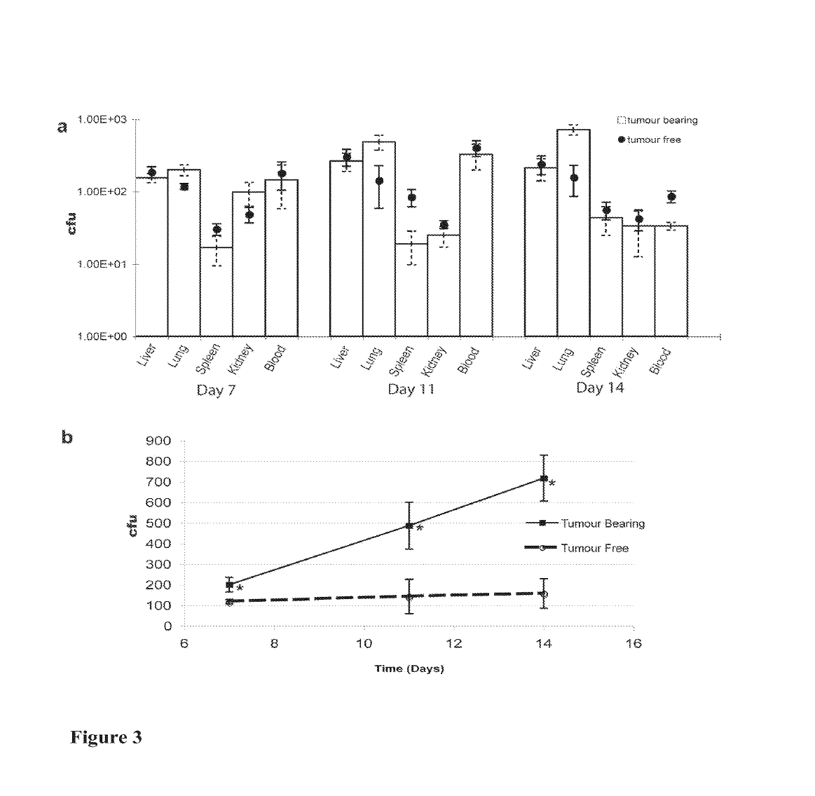

Bacterial luminescence was readily detected specifically in s.c. tumours by live whole body imaging (IVIS) (FIG. 1b). Ex vivo bacterial quantification by culture of tumours and other organs showed presence of UCC2003 in all tissues examined, indicating migration of B. breve from the GIT and systemic spread to internal tissues. Numbers of UCC2003 were low in organs, ≦100 colony forming units (cfu)/organ, relative to the bacterial numbers present in tumour, and decreased over time. In contrast, high-level replication was observed in tumours, reaching cfu levels of 10⁶/tumour by day 14 (FIG. 1b). Tumour volume curves indicated no significant difference in tumour growth rates between bifidobacterial administered mice and controls (data not shown). Live imaging of luminescence from lux-expressing bifidobacteria provided a robust system for indication of bacterial numbers in tumour masses as well as providing evidence of plasmid gene expression (FIG. 4).

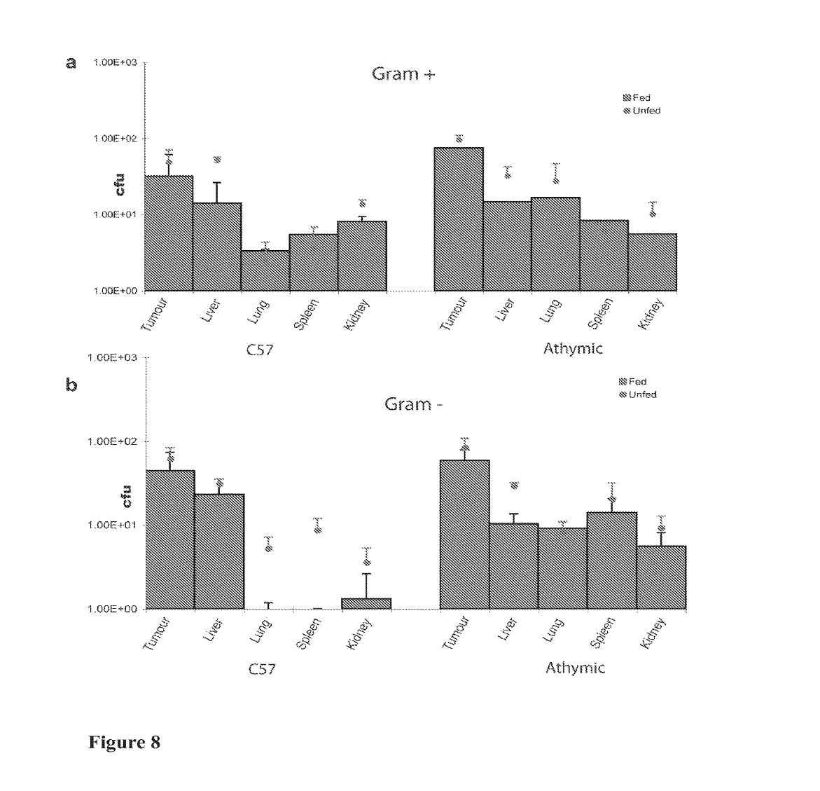

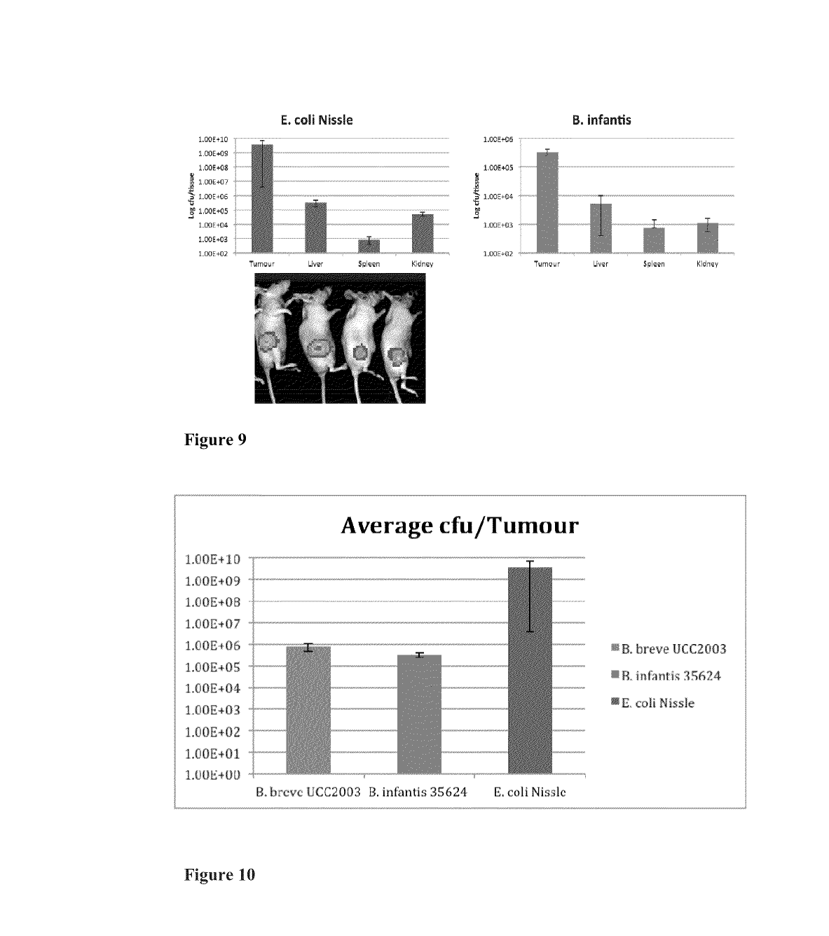

Similar results were obtained with E. coli Nissle 1917 engineered with the plasmid p16Slux which contains the constitutive P_{HELP}luxABCDE operon⁵⁹, orally administered to athymic mice bearing s.c. RENCA tumours (FIG. 9).

B. breve UCC2003 Translocates from the GIT and Colonises Tumours when Administered Orally at Levels Equal to I.V. Administration

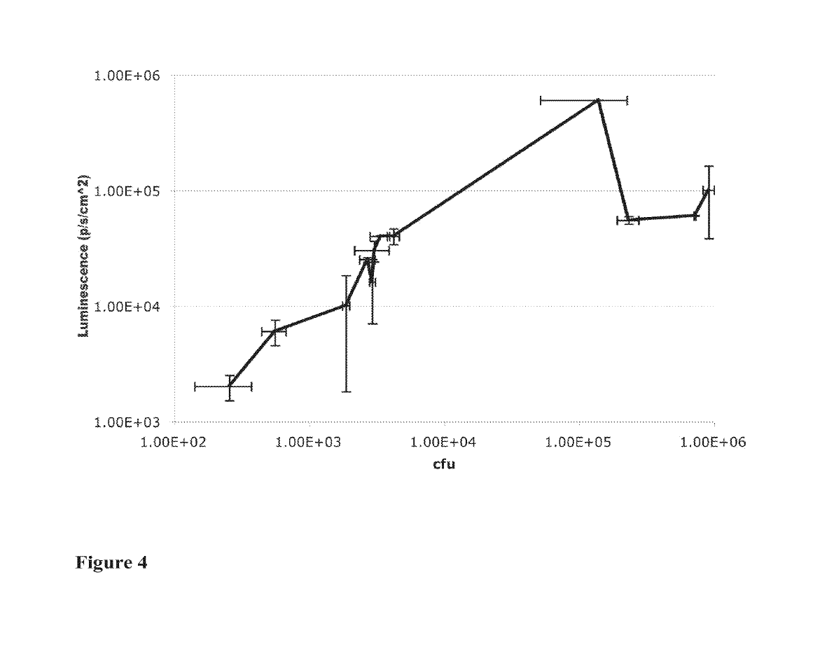

Previous studies have reported bacterial localisation to tumours only following intravenous administration. When intravenous administration of this strain was examined, similar results were observed (FIG. 1c), with no significant differences in colonisation levels between oral and iv administration in tumours or other tissues (FIG. 1d, FIG. 5), indicating that oral administration is as effective in achieving targeted tumour growth as the well described intravenous route, using our optimised protocols.

Comparisons indicated that oral administration was as effective in achieving targeted tumour-located growth as the well-described i.v. route, using our optimised protocols, and indeed, maximum tumour cfu levels were observed with orally administered mice, on day 14 (FIG. 1d). No significant differences were observed between oral and i.v. administration in organs (p>0.058) (FIG. 5). In order to establish that the phenomenon was not tumour-type specific, the inventors repeated the study in MF1 nu/nu mice bearing s.c. MCF7 human breast tumour xenografts or C57 mice bearing s.c. Lewis Lung Carcinoma tumours and observed similar results.

### Trafficking of Orally-Administered Bacteria to Patient Tumours

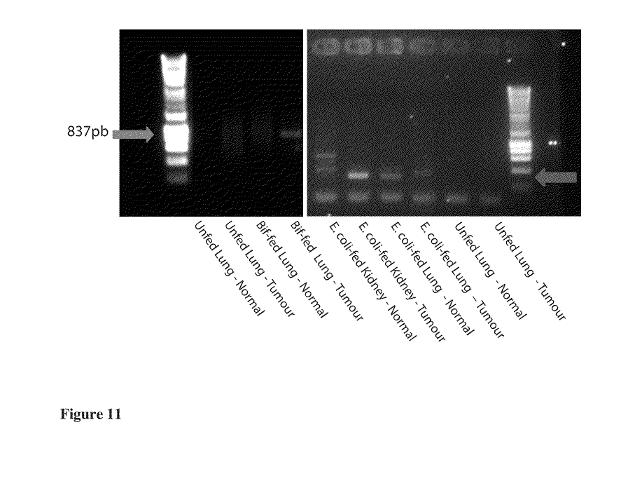

PCR validated the presence of E. coli Nissle 1917 or Bifidobacterium 35624 within the tissue of patients orally administered the relevant strain (FIG. 11).

Pattern of B. breve UCC2003 Growth within Tumours

UCC2003 levels within central or peripheral regions of subcutaneous tumours were assessed utilising quantitative PCR (specific for lux) on total DNA isolated from tissue taken from either the periphery or centre of 1 cm³ tumours resected from athymic mice 11 days post feeding. Vector was detectable only in the central region, at 7.28×10⁸ (±1.87×10⁷) bacterial cells/μg total DNA, suggesting preferential growth within anaerobic regions (data not shown).

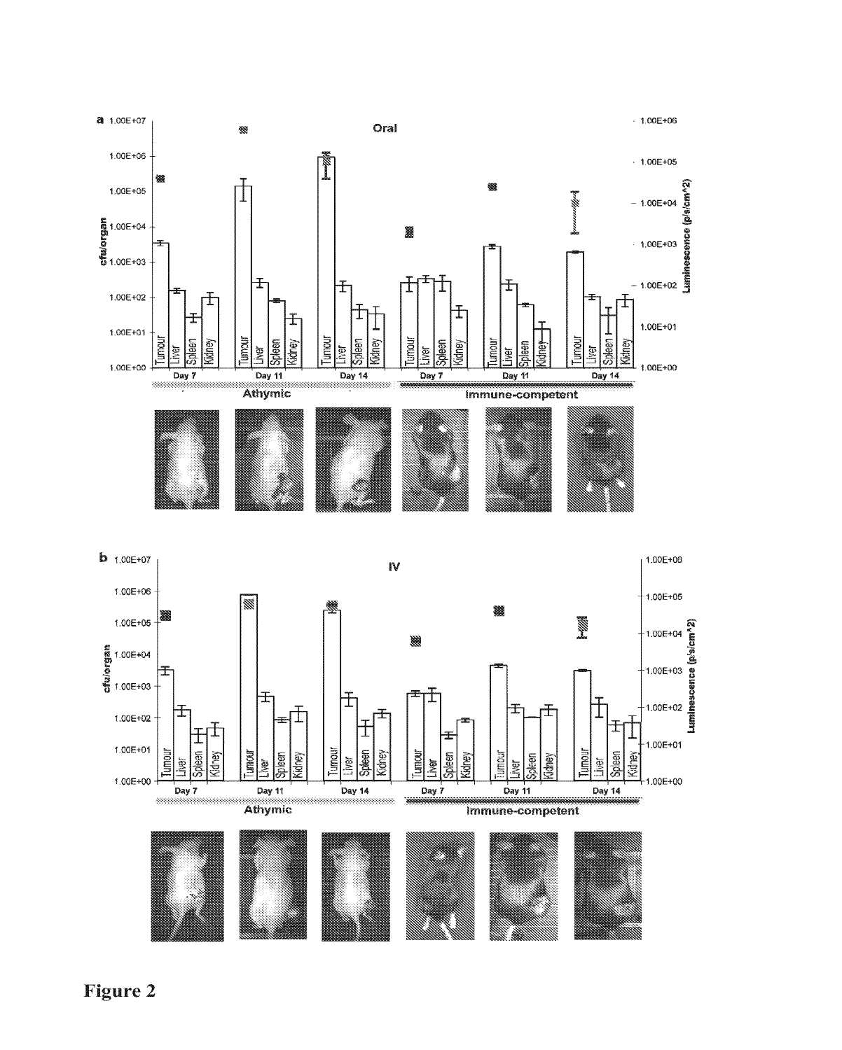

The B16 tumour model spontaneously metastasises from s.c. tumours to lungs. Significantly increasing bacterial load was observed over time in lungs of tumour bearing athymic mice fed UCC2003 (p<0.045), indicating bifidobacterial colonisation and replication in small (potentially pre-hypoxic) metastatic nodules (FIG. 3(b)). No increase in luminescence was observed, suggesting these bacterial levels were below the luminescence detection limit.

### Route of Trafficking is Via Serum

UCC2003 was recovered from serum and not the cellular component of blood up to day 14 post feeding (FIG. 5), indicating haematogenous trafficking of free bacteria from the GIT. B. breve numbers in blood were similar at all time points whether administered i.v. or orally.

Translocation does not Require Presence of Tumour

When cfu levels in blood and organs were compared between tumour-bearing and tumour-free mice, no significant differences were observed for liver, kidney and blood (p>0.240) (FIG. 3a), indicating that translocation is not mediated or promoted by some tumour-related factor. The B16 tumour model spontaneously metastasises from s.c. tumours to lungs. Significantly increasing bacterial load was observed over time in lungs of tumour bearing athymic mice (p<0.045), indicating bifidobacterial colonisation and replication in metastatic nodules. No increase in luminescence was observed, suggesting these bacterial levels were below the luminescence detection limit. Nonetheless, live imaging of luminescence from lux expressing bifidobacteria provided a robust system for indication of bacterial numbers in larger tumour masses as well as providing evidence of plasmid gene expression (FIG. 3b).

### Immune Responses to Vector

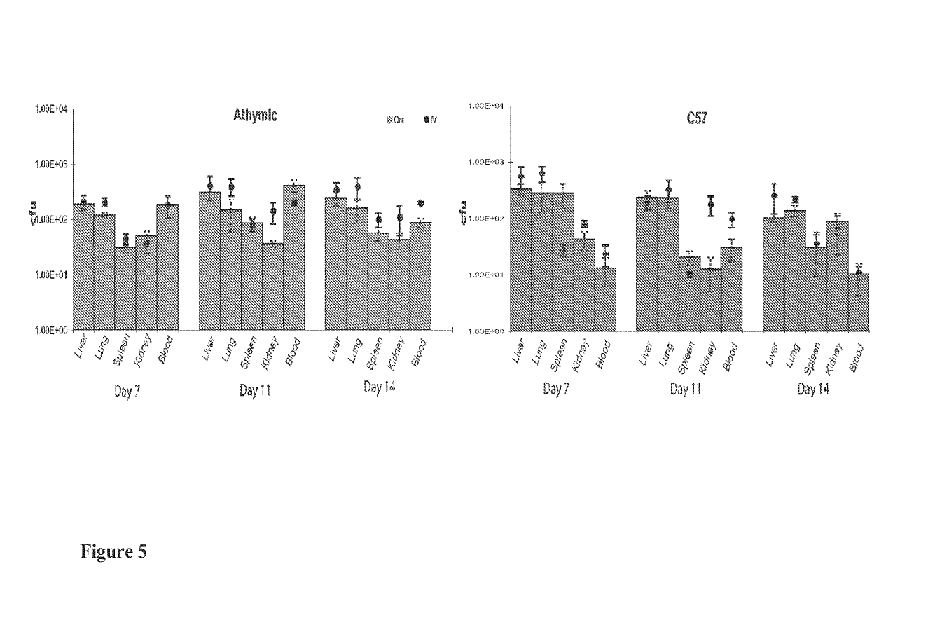

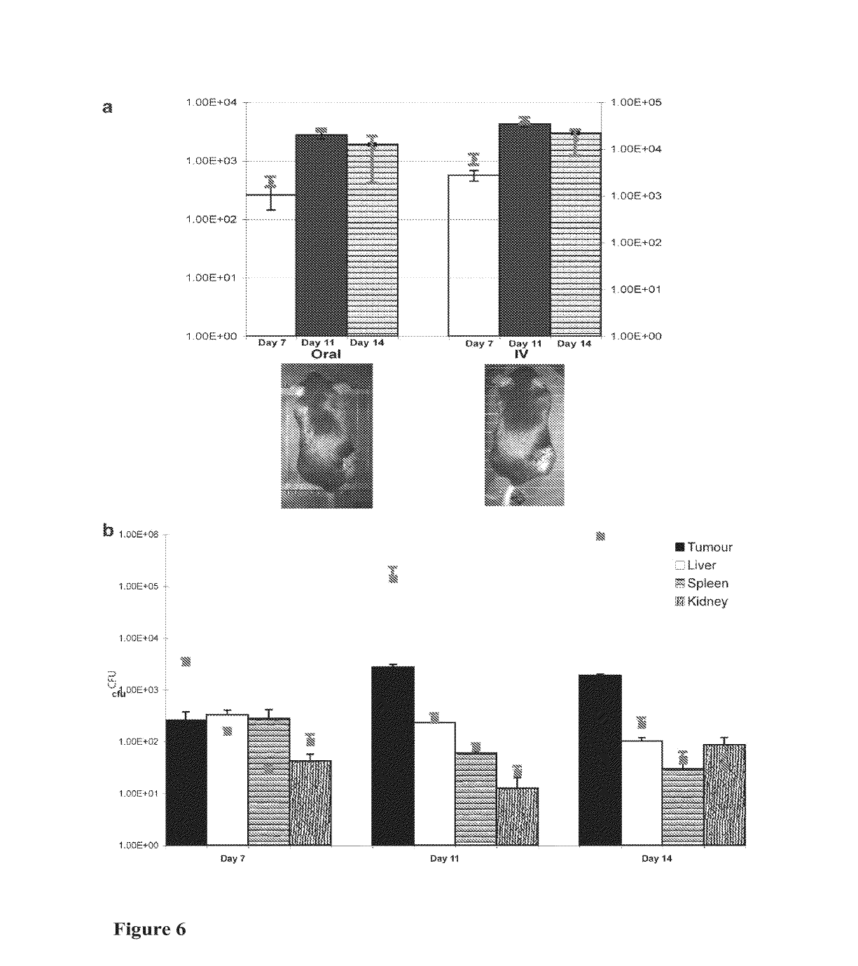

Comparison between bacterial levels in athymic and immunocompetent C57 mice indicated the involvement of T cells in bifidobacterial containment in tumours, while translocation to organs occurred at similar levels (p>0.107) (FIG. 6). UCC2003 levels in tumours were significantly increased specifically in tumours in the absence of T cells, with both oral and i.v. administration. To investigate the effect of UCC2003 on T cell levels in tumours, flow cytometry analysis was used to quantify CD3-positive T cells in s.c. tumours from C57 mice orally administered UCC2003 or PBS (FIG. 7). A 25.8 (+/−5.8) % increase in total T cell numbers was observed in UCC2003 colonised tumours 11 days post feeding, compared with unfed mice (p=0.06, n=3). Nonetheless, even in immunocompetent animals, bacteria were readily detected by IVIS imaging specifically in tumours at levels comparable to i.v. administration (FIG. 6(a)). The finding that organ cfu levels were not higher in athymic mice suggests that T cells do not inhibit translocation of bifidobacteria, but do significantly inhibit tumour-associated replication locally.

UCC2003 was found to persist indefinitely in athymic mice (up to 40 cfu/organ and 5 cfu/ml blood at week 5-post feeding) but was eventually cleared in immunocompetent animals by week 5-post oral administration (data not shown), indicating immune recognition by T cells but the ability to evade other immune effector systems. IgG antibody specific for UCC2003 was detected by ELISA in serum taken from immune-competent C57 mice 14 days post administration (FIG. 6b). There was no significant difference between anti-UCC2003 IgG antibody levels in serum from IV and fed mice (p=0.359). Circulating levels of cytokines examined were not significantly increased in serum from fed mice, except for the proinflammatory cytokine IL113 (946.16±47.31% with respect to unfed mice levels, p<0.001), suggesting the presence of some, but not a dramatic, degree of immune inflammatory response to systemic UCC2003 cells (data not shown).

### Examination of Mechanism of Gastrointestinal Egress

Increased levels of cytokines such as IFNγ in the GIT are associated with increases in epithelial permeability and gut barrier loss in pathogenic bacterial sepsis^{19,20}. Non-pathogenic bacteria have previously been shown to upregulate cytokines such as IFNγ and IL12 in the GIT^{21,22}. In our experiments, cytokine analyses of IFNγ, IL1b, IL12p70, IL6, IL10, TNFα and KC levels were performed on GIT samples from UCC2003-fed and PBS-fed B16 tumour bearing athymic mice. Only IFNγ levels differed between the groups, with UCC2003 colonised GIT samples found to have significantly higher levels of this cytokine (295±44%, p=0.003), with respect to unfed mice, suggesting a possible IFNγ induced increase in epithelial permeability.

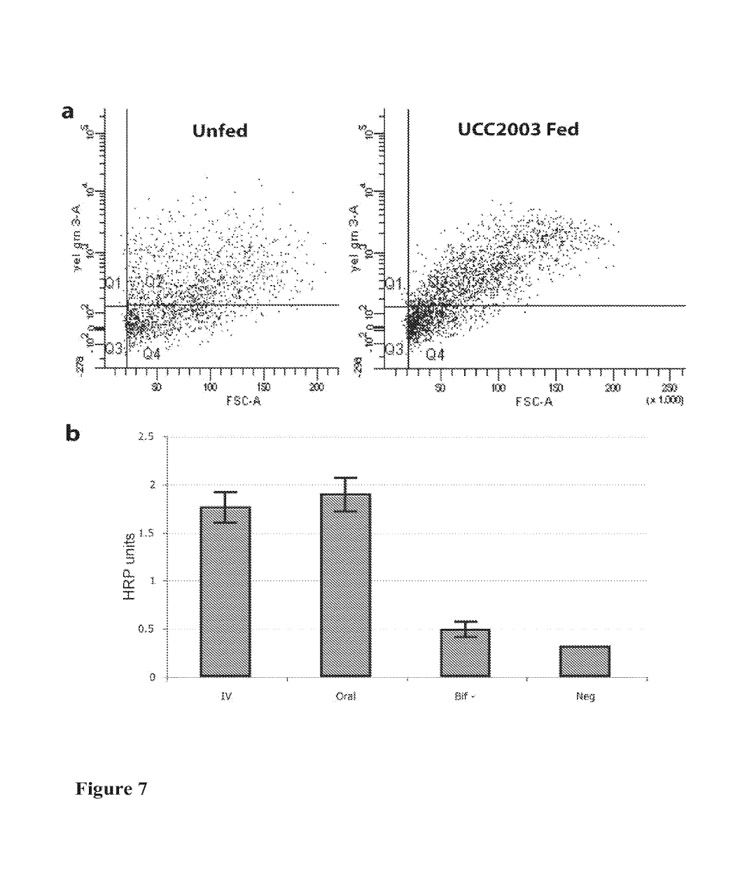

In order to examine for the possibility that UCC2003 feeding might result in generic increased translocation of GIT-residing bacteria, we compared systemic bacterial levels between UCC2003 fed and PBS fed mice. Low levels (5-50 cfu) of indigenous commensal bacteria (subsequently identified as lactobacilli, enterobacteriaceae, staphlococci, micrococci and Bacteroides by API analysis) were recovered from all organs examined, but not blood, in both immunocompetent and athymic mice (FIG. 8). No differences in non-UCC2003 bacterial levels were observed between UCC2003-fed and control (PBS-fed) mice (p>0.114), except for decreases in Gram-negative bacteria in lung, liver and kidney of C57 mice fed UCC2003 (p<0.044), indicating inhibition of certain species. These data suggest that UCC2003 specifically mediates its own translocation, with no collateral translocation of other commensal bacteria.

To examine the route of bacterial trafficking from the GIT, various fractions of whole blood were assessed for presence of B. breve. Up to 500 cfu/ml UCC2003 were recovered from serum and not the cellular component of blood, when examined up to day 14 post feeding, indicating haematogenous trafficking of free bacteria from the GIT, rather than immune cell-mediated phagocytosis and transport. Orally administered B. breve numbers in blood remained static over this 14-day period, and were similar to i.v. levels at day 7 (p=0.296), while i.v. UCC2003 numbers decreased over time.

We also assessed if the presence of a tumour was required for bacterial translocation to occur. When cfu levels in blood and organs were compared between tumour-bearing and tumour-free mice, no significant differences were observed for liver, kidney and blood (p>0.240) (FIG. 3(a)), indicating that translocation is not mediated or promoted by some tumour-related factor.

B. breve Colonisation of and Replication in Tumours is Increased Specifically in Tumours in the Absence of T Cells, with Both Oral and I.V. Administration

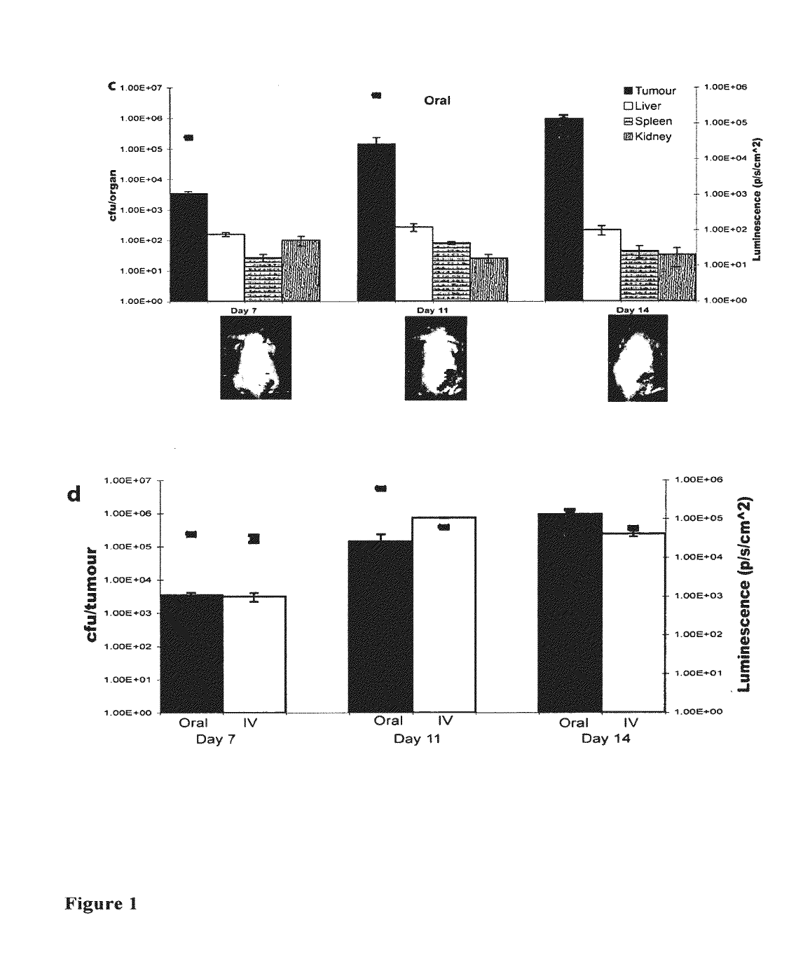

Comparison between bacterial levels in athymic and immunocompetent C57 mice indicated the involvement of T cells in bifidobacterial containment in tumours, while translocation to organs occurred at similar levels (p>0.107) (FIG. 2). UCC2003 levels in tumours were significantly increased specifically in tumours in the absence of T cells, with both oral and i.v. administration (FIG. 2). Nonetheless, even in immunocompetent animals, bacteria were detected by IVIS imaging specifically in tumours, indicating the potential for this strategy of tumour detection or treatment. The finding that organ cfu levels were not higher in athymic mice suggests that T cells do not inhibit translocation of bifidobacteria, but do significantly inhibit tumour-associated replication locally.

### Discussion

The inventors have shown for the first time that non-invasive bacteria can traffic to and proliferate with distal tissues following ingestion. Furthermore, such bacteria can mediate high-level gene expression over time in systemic tumours in murine models.

The inventors have shown that this phenomenon is not unique to a single strain of bacterium, validating similar findings with three species of bacteria, featuring both Gram-positive and Gram-negative genera, as well as facultatively anaerobic and strictly anaerobic species.

Importantly, the inventors have recapitulated the original findings from mice, in human cancer patients, with two genera. E. coli Nissle 1917 detected in tumour tissue from fed patients, and not in unfed, indicating that the original findings in mice can be recapitulated in humans. Similarly, Bifidobacterium 35624 was detected in tumours of fed patients. For E. coli Nissle 1917, tissue samples from non-malignant tissue adjacent to tumours of fed patients were also positive by PCR, and currently it is unclear whether this represents the genuine presence of viable bacteria in non-tumour tissue, or DNA contamination from the tumour.

The use of bacteria expressing lux in tumours presents a potentially powerful diagnostic clinical tool. Furthermore, the bacteria could be engineered to express alternative genes for use with existing clinical diagnostic equipment, such as HSV thymidine kinase in combination with positron emission tomography (PET)²³.

UCC2003 preferentially colonises the caecum, with peak levels of 10⁶ cfu/g tissue observed 19 days post feeding, and 10⁵ cfu/g tissue one month post feed¹⁸. It is unlikely that increasing tumour bacterial load over time is due to newly translocated bacteria becoming trapped in tumour microvasculature, as bacterial levels also increased in tumours in i.v. administered mice receiving a single dose of UCC2003. The precise mechanism of tumour-specific bacterial growth has yet to be demonstrated. Several groups have demonstrated homing to and replication in tumours, with many types of bacteria, including bifidobacteria, following i.v. injection^{1-3,5}. Early observations with strictly anaerobic bacteria (clostridia and bifidobacteria) lead to the hypothesis that, unlike normal tissues, the hypoxic environment in tumours provides anaerobic growth conditions^{24,25}. However, evidence of similar tumour-specific growth of non-anaerobic bacteria, coupled with findings of growth in small, pre-hypoxic tumours, suggest that lack of oxygen in the necrotic centre of tumours may not be the determining factor for the tumour specific nature of bacterial growth in theses settings⁵. In the inventors' studies, B. breve UCC2003 was observed to increase over time in lungs containing small (<5 mm³) B16 pulmonary metastatic nodules, also suggesting that hypoxia may not be a requirement for anaerobic bacterial targeting. Yu et at proposed that the entry, survival and replication of bacteria in tumours is dependent on tumour vascularisation and the tumour immune microenvironment which provides a sanctuary for a small number of bacteria that will escape the immune system⁵. This model involves bacteria entering the tumour's leaky vasculature and escaping the host immunosurveillance due to the immune-privileged nature of solid tumours. Tumour cell line xenografts are known to have different vasculature to spontaneously arising tumours, and investigations in an appropriate spontaneous tumour murine model may yield more clinically relevant data. The nutrient rich environment may also play an important role as evidenced by findings that tumours can support the replication of auxotrophic strains of S. typhimurium^{6,26,27}. The nature of growth within tumours also appears to be bacterial strain specific. It has been reported for many species, including strains of S. typhimurium, that the bacterial vector growth was confined to the central necrotic regions of tumours while in contrast, the S. typhimurium A1 strain has been shown to grew throughout the tumour, including viable malignant tissue in a wide range of tumour models^{6,26-30}. In the inventors' studies, UCC2003 was detected solely in the central necrotic region.

The inventors report that UCC2003 persisted indefinitely in athymic mice but was eventually cleared in immunocompetent animals indicating immune recognition by T cells but the ability to evade other immune effector systems. It is noteworthy that bacterial replication was restricted in immune-competent mice specifically in tumours. It is likely that these anti-UCC2003 immune responses are active systemically, but a reduction in organ numbers as evidence by the inventors' assays may be masked by the constant influx of newly translocated bacteria, while tumour located bacteria do not exponentially multiply at the same rate in tumours in immune-competent as in athymic mice, resulting in readily measurable differences. The absence of significant up-regulation of cytokines characteristic of anti-bacterial responses in blood further demonstrated immune tolerance to this strain. Previous studies in cynomolgus monkeys, dogs and guinea pigs have indicated no adverse effects following intravenous administration of Bifidobacterium longum³¹. Recent reports indicated differential stimulation of the immune system by bifidobacteria depending on species, and indeed strain, with B. breve shown to have little effect on the immune system, while specific B. longum strains influenced the orientation of Th1/Th2 responses differently³².

Fu et at previously described the use of B. longum to deliver a therapeutic peptide to the GIT³³. In that report, B. longum expressing endostatin was administered orally to athymic mice, and the authors reported that subsequent gut absorption of the therapeutic peptide resulted in slowing in the growth of s.c. liver tumours. Translocation of bacteria from the gut to tumours was not investigated or reported in that study. The current inventors demonstrated that B. breve UCC2003 survives the upper GIT, colonises the caecum and translocates to extra-intestinal sites. Most bacteria which breach the epithelial barrier are killed by gut-associated lymphoid tissue¹⁴. In healthy animal models in which the intestinal barrier is not physically damaged, indigenous bacteria have been shown to translocate by an intracellular route through the epithelial cells lining the intestines and then travel via the lymph to the Mesenteric Lymph Nodes^{34,35}. The inventors' data suggest that immune cell uptake or transport was not involved in UCC2003 trafficking as all B. breve bacteria in blood were found in serum only. In animal models exhibiting damage to the mucosal epithelium, indigenous bacteria translocate intercellularly between the epithelial cells to directly access the blood¹⁵. Most bacteria, which breach the epithelial barrier, are killed by the gut-associated lymphoid tissue GALT³³. It is unknown why UCC2003 was tolerated by the immune system in these studies. It was found that this bacterium persisted indefinitely in athymic mice (up to 40 cfu/organ and 5 cfu/ml blood at week 5; data not shown) but was eventually cleared in immunocompetent animals by week 5-post oral administration, indicating immune recognition by T cells but the ability to evade other immune effector systems. Findings of 1000 times increase in tumour associated bacterial levels in the absence of T cells presents the potential for improvement of this strategy via temporary local manipulation of T cells.

The ability of microorganisms to translocate, survive, and proliferate in extra-intestinal tissues involves complex interactions between the host defense mechanisms and the bacterium's ability to invade host tissues. Although the importance of host immune function^{34,36,37} and the bacterium's intestinal population size^{34,39-41} have been implicated as significant contributory factors, the precise mechanisms involved remains unknown^{42,43}.

It is unlikely that increasing tumour bacterial load over time is due to newly translocated bacteria becoming trapped in tumour microvasculature, as bacterial levels also increased in tumours in i.v. administered mice receiving a single dose of UCC2003.

While significant evidence for the translocation of pathogenic bacteria exists^{44,45}, relatively little information is available on translocation by indigenous species. In animal models, the recovery of Bacteroides, lactobacilli and enterococci has been reported in healthy pathogen-free mice^{15,39,38,46}, and Yamazaki et al. also reported B. longum colonisation of organs post feeding⁴⁷. Sampling from humans has indicated that bacterial translocation may be a phenomenon that occurs in healthy individuals and may be a normal physiological event without deleterious consequences⁴⁸. Clinical studies with probiotic bacteria conducted with healthy subjects have not reported severe disease caused by the bacteria even when shown to translocate from the gastrointestinal tract⁴². Lactobacillus, Leuconostoc, Pediococcus, Enterococcus, and Bifidobacterium have been isolated from infected lesions in patients^{42, 49-51}. Penn et al.³⁴ reported an increased translocation from the GIT of S-180 tumour-bearing mice, leading to the hypothesis that immune deficiencies associated with progressive tumour growth may be sufficient to permit viable bacteria to translocate from the GIT. The inventors did not observe statistically significant differences in Gram-negative or Gram-positive organ bacterial levels between B16 tumour-bearing and -free athymic or C57 mice, and the presence of tumour was not necessary for UCC2003 translocation to systemic organs to occur.

The population level obtained in the GIT by a particular bacterial species may be a critical factor determining whether or not this bacterial species will translocate to other organs³⁸. This has been observed for E. coli in the presence of an intact anaerobic flora, where caecal concentrations of greater than 10⁷ E. coli per gram are required before this organism reliably translocates to other sites in healthy mice³⁹⁻⁴¹. The inventors' cytokine analyses indicated that UCC2003 colonisation altered the local GIT cytokine milieu, perhaps leading to an IFNγ-induced increase in epithelial permeability. Examination of other commensal bacteria indicate that rather than B. breve inducing a general leakiness of the GIT barrier permitting generic bacterial translocation, UCC2003 specifically mediates its own translocation, with no collateral translocation of other commensal bacteria.

The B. breve UCC2003 translocated bacteria proved non-pathogenic even in immunocompromised animals and if desired the vector could be easily cleared systemically by antibiotic administration. The current inventors have shown that ingestion of these non-pathogenic bacteria carrying a gene of interest, in this case a luminescent reporter, results in high-level expression specifically in tumours. Since up to 10⁶ bacterial cells (with multiple plasmid gene copies/cell) were recovered from tumours, the potential for efficient delivery of genes to the tumour environment compares favourably with existing vectors. The current inventors have shown that bifidobacteria and E. coli survive the upper GIT, colonise the caecum and translocate, and that these translocated bacteria are non-pathogenic, even in immunocompromised animals. They have shown that ingestion of these food-grade bacteria carrying a gene of interest results in high-level expression specifically in tumours.

Potential for strain optimisation exists through re-isolating vector with increased efficiencies from experimental tumours, as has been accomplished with other tumour targeting bacteria¹³⁻¹⁵. Overall, this strategy represents a novel, safe and non-invasive vector system, with the potential to deliver therapeutic or diagnostic agents systemically. The vector could be cleared by antibiotic administration. The current invention could also be used in non-cancer related physiological studies as a reporting system for investigations on translocation and related effects, especially since translocation to organs occurred at the same level in the absence or presence of tumour. The route of administration of a therapeutic is important in clinical and commercial settings. Direct intratumoural administration of bacterial vectors restricts usage to accessible tumours, and while intravenous administration facilitates targeting of systemic tumour sites, the oral route is amenable to administering much higher doses of bacteria safely and is likely to be more appealing when applied to the clinical setting due to ease of drug application. Furthermore, pharmaceutical industry dogma displays a preference for the oral route of administration.

The words “comprises/comprising” and the words “having/including” when used herein with reference to the present invention are used to specify the presence of stated features, integers, steps or components but does not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

### REFERENCES

1. Fujimori M, Amano J, Taniguchi S. The genus Bifidobacterium for cancer gene therapy. Curr Opin Drug Discov Devel 2002; 5(2): 200-203.
2. Li Z, Fallon J, Mandeli J, Wetmur J, Woo S L. A genetically enhanced anaerobic bacterium for oncopathic therapy of pancreatic cancer. J Natl Cancer Inst 2008; 100(19): 1389-1400.
3. Vassaux G, Nitcheu J, Jezzard S, Lemoine N R. Bacterial gene therapy strategies. J Pathol 2006; 208(2): 290-298.
4. Wei M Q, Mengesha A, Good D, Anne J. Bacterial targeted tumour therapy-dawn of a new era. Cancer Lett 2008; 259(1): 16-27.
5. Yu Y A, Shabahang S, Timiryasova T M, Zhang Q, Beltz R, Gentschev I et al. Visualization of tumors and metastases in live animals with bacteria and vaccinia virus encoding light-emitting proteins. Nat Biotechnol 2004; 22(3): 313-320.
6. Zhao M, Yang M, Li X M, Jiang P, Baranov E, Li S et al. Tumor-targeting bacterial therapy with amino acid auxotrophs of GFP-expressing Salmonella typhimurium. Proc Natl Acad Sci USA 2005; 102(3): 755-760.
7. Morrissey D, O'Sullivan, G C, Tangney, M. Tumour targeting with systemically administered bacteria. Curr Gene Ther 2010; 10(1).
8. Tangney M, Gahan, C G M. Listeria monocytogenes as a vector for anti-cancer therapies. Curr Gene Ther 2010; 10(1).
9. van Pijkeren J, Morrissey D, Cronin M, Rajendran S, O'Sullivan G, Gahan C et al. A novel Listeria monocytogenes-based DNA delivery system for cancer gene therapy. Human Gene Therapy 2010; 21(3).
10. Leahy S C, Higgins D G, Fitzgerald G F, van Sinderen D. Getting better with bifidobacteria. J Appl Microbiol 2005; 98(6): 1303-1315.
11. Li X, Fu G F, Fan Y R, Liu W H, Liu X J, Wang J J et al. Bifidobacterium adolescentis as a delivery system of endostatin for cancer gene therapy: selective inhibitor of angiogenesis and hypoxic tumor growth. Cancer Gene Ther 2003; 10(2): 105-111.
12. Nakamura T, Sasaki T, Fujimori M, Yazawa K, Kano Y, Amano J et al. Cloned cytosine deaminase gene expression of Bifidobacterium longum and application to enzyme/pro-drug therapy of hypoxic solid tumors. Biosci Biotechnol Biochem 2002; 66(11): 2362-2366.
13. Yi C, Huang Y, Guo Z Y, Wang S R. Antitumor effect of cytosine deaminase/5-fluorocytosine suicide gene therapy system mediated by Bifidobacterium infantis on melanoma. Acta Pharmacol Sin 2005; 26(5): 629-634.
14. Balzan S, de Almeida Quadros C, de Cleva R, Zilberstein B, Cecconello I. Bacterial translocation: overview of mechanisms and clinical impact. J Gastroenterol Hepatol 2007; 22(4): 464-471.
15. Berg R D. Bacterial translocation from the gastrointestinal tract. Adv Exp Med Biol 1999; 473: 11-30.
16. Ishibashi N, Yamazaki S. Probiotics and safety. Am J Clin Nutr 2001; 73(2 Suppl): 465S-470S.
17. Riedel C U, Monk I R, Casey P G, Morrissey D, O'Sullivan G C, Tangney M et al. Improved luciferase tagging system for Listeria monocytogenes allows real-time monitoring in vivo and in vitro. Appl Environ Microbiol 2007; 73(9): 3091-3094.
18. Cronin M, Sleator R D, Hill C, Fitzgerald G F, van Sinderen D. Development of a luciferase-based reporter system to monitor Bifidobacterium breve UCC2003 persistence in mice. BMC Microbiol 2008; 8: 161.
19. Luyer M D, Buurman W A, Hadfoune M, Wolfs T, van't Veer C, Jacobs J A et al. Exposure to bacterial DNA before hemorrhagic shock strongly aggravates systemic inflammation and gut barrier loss via an IFN-gamma-dependent route. Ann Surg 2007; 245(5): 795-802.
20. McKay D M, Watson J L, Wang A, Caldwell J, Prescott D, Ceponis P M et al. Phosphatidylinositol 3′-kinase is a critical mediator of interferon-gamma-induced increases in enteric epithelial permeability. J Pharmacol Exp Ther 2007; 320(3): 1013-1022.
21. Haller D, Blum S, Bode C, Hammes W P, Schiffrin E J. Activation of human peripheral blood mononuclear cells by nonpathogenic bacteria in vitro: evidence of NK cells as primary targets. Infect Immun 2000; 68(2): 752-759.
22. Haller D, Bode C, Hammes W P, Pfeifer A M, Schiffrin E J, Blum S. Non-pathogenic bacteria elicit a differential cytokine response by intestinal epithelial cell/leucocyte co-cultures. Gut 2000; 47(1): 79-87.
23. Iyer M, Sato M, Johnson M, Gambhir S S, Wu L. Applications of molecular imaging in cancer gene therapy. Curr Gene Ther 2005; 5(6): 607-618.
24. Lemmon M J, van Zijl P, Fox M E, Mauchline M L, Giaccia A J, Minton N P et al. Anaerobic bacteria as a gene delivery system that is controlled by the tumor microenvironment. Gene Ther 1997; 4(8): 791-796.
25. Yazawa K, Fujimori M, Amano J, Kano Y, Taniguchi S. Bifidobacterium longum as a delivery system for cancer gene therapy: selective localization and growth in hypoxic tumors. Cancer Gene Ther 2000; 7(2): 269-274.
26. Nagakura C, Hayashi K, Zhao M, Yamauchi K, Yamamoto N, Tsuchiya H et al. Efficacy of a genetically-modified Salmonella typhimurium in an orthotopic human pancreatic cancer in nude mice. Anticancer Res 2009; 29(6): 1873-1878.
27. Zhao M, Yang M, Ma H, Li X, Tan X, Li S et al. Targeted therapy with a Salmonella typhimurium leucine-arginine auxotroph cures orthotopic human breast tumors in nude mice. Cancer Res 2006; 66(15): 7647-7652.
28. Hayashi K, Zhao M, Yamauchi K, Yamamoto N, Tsuchiya H, Tomita K et al. Cancer metastasis directly eradicated by targeted therapy with a modified Salmonella typhimurium. J Cell Biochem 2009; 106(6): 992-998.
29. Hayashi K, Zhao M, Yamauchi K, Yamamoto N, Tsuchiya H, Tomita K et al. Systemic targeting of primary bone tumor and lung metastasis of high-grade osteosarcoma in nude mice with a tumor-selective strain of Salmonella typhimurium. Cell Cycle 2009; 8(6): 870-875.
30. Zhao M, Geller J, Ma H, Yang M, Penman S, Hoffman R M. Monotherapy with a tumor-targeting mutant of Salmonella typhimurium cures orthotopic metastatic mouse models of human prostate cancer. Proc Natl Acad Sci USA 2007; 104(24): 10170-10174.
31. Fujimori M. Genetically engineered bifidobacterium as a drug delivery system for systemic therapy of metastatic breast cancer patients. Breast Cancer 2006; 13(1): 27-31.
32. Menard O, Butel M J, Gaboriau-Routhiau V, Waligora-Dupriet A J. Gnotobiotic mouse immune response induced by Bifidobacterium sp. strains isolated from infants. Appl Environ Microbiol 2008; 74(3): 660-666.
33. Fu G F, Li X, Hou Y Y, Fan Y R, Liu W H, Xu G X. Bifidobacterium longum as an oral delivery system of endostatin for gene therapy on solid liver cancer. Cancer Gene Ther 2005; 12(2): 133-140.
34. Penn R L, Maca R D, Berg R D. Increased translocation of bacteria from the gastrointestinal tracts of tumor-bearing mice. Infect Immun 1985; 47(3): 793-798.
35. Penn R L, Nguyen V Q, Specian R D, Stevens P, Berg R D. Interleukin-2 enhances the translocation of Escherichia coli from the intestines to other organs. J Infect Dis 1991; 164(6): 1168-1172.
36. Berg R D. Translocation of enteric bacteria in health and disease. Curr Stud Hematol Blood Transfus 1992; (59): 44-65.
37. Owens W E, Berg R D. Bacterial translocation from the gastrointestinal tract of athymic (nu/nu) mice. Infect Immun 1980; 27(2): 461-467.
38. Berg R D, Garlington A W. Translocation of certain indigenous bacteria from the gastrointestinal tract to the mesenteric lymph nodes and other organs in a gnotobiotic mouse model. Infect Immun 1979; 23(2): 403-411.
39. Berg R D, Owens W E Inhibition of translocation of viable Escherichia coli from the gastrointestinal tract of mice by bacterial antagonism. Infect Immun 1979; 25(3): 820-827.
40. Steffen E K, Berg R D. Relationship between cecal population levels of indigenous bacteria and translocation to the mesenteric lymph nodes. Infect Immun 1983; 39(3): 1252-1259.
41. Steffen E K, Berg R D, Deitch E A. Comparison of translocation rates of various indigenous bacteria from the gastrointestinal tract to the mesenteric lymph node. J Infect Dis 1988; 157(5): 1032-1038.
42. Liong M T. Safety of probiotics: translocation and infection. Nutr Rev 2008; 66(4): 192-202.
43. Rodriguez A V, Baigori M D, Alvarez S, Castro G R, Oliver G. Phosphatidylinositol-specific phospholipase C activity in Lactobacillus rhamnosus with capacity to translocate. FEMS Microbiol Lett 2001; 204(1): 33-38.
44. van der Waaij D, Berghuis-de Vries J M, Lekkerkerk-van der W. Colonization resistance of the digestive tract and the spread of bacteria to the lymphatic organs in mice. J Hyg (Lond) 1972; 70(2): 335-342.
45. Wells C L. Relationship between intestinal microecology and the translocation of intestinal bacteria. Antonie Van Leeuwenhoek 1990; 58(2): 87-93.
46. Berg R D. Bacterial translocation from the intestines. Jikken Dobutsu 1985; 34(1): 1-16.
47. Yamazaki S, Machii K, Tsuyuki S, Momose H, Kawashima T, Ueda K. Immunological responses to monoassociated Bifidobacterium longum and their relation to prevention of bacterial invasion. Immunology 1985; 56(1): 43-50.
48. Sedman P C, Macfie J, Sagar P, Mitchell C J, May J, Mancey-Jones B et al. The prevalence of gut translocation in humans. Gastroenterology 1994; 107(3): 643-649.
49. Pavan S, Desreumaux P, Mercenier A. Use of mouse models to evaluate the persistence, safety, and immune modulation capacities of lactic acid bacteria. Clin Diagn Lab Immunol 2003; 10(4): 696-701.
50. Tancrede C H, Andremont A O. Bacterial translocation and gram-negative bacteremia in patients with hematological malignancies. J Infect Dis 1985; 152(1): 99-103.
51. Zhou J S, Shu Q, Rutherfurd K J, Prasad J, Gopal P K, Gill H S. Acute oral toxicity and bacterial translocation studies on potentially probiotic strains of lactic acid bacteria. Food Chem Toxicol 2000; 38(2-3): 153-161.
52. Riedel C U M I, Casey P G, Morrissey D, O'Sullivan G C, Tangney M, Hill C, Gahan C G. Improved luciferase tagging system for Listeria monocytogenes allows real-time monitoring in vivo and in vitro. Appl Environ Microbiol 2007; 2007; 73:3091-3094. doi: 10.1128/AEM.02940-06. [PubMed].
53. Simpson P J, Stanton C, Fitzgerald G F, Ross R P. Genomic diversity and relatedness of bifidobacteria isolated from a porcine cecum. J Bacteriol 2003; 185(8): 2571-2581.
54. Turroni F, Foroni E, Pizzetti P, Giubellini V, Ribbera A, Merusi P et al. Exploring the diversity of the bifidobacterial population in the human intestinal tract. Appl Environ Microbiol 2009; 75(6): 1534-1545.
55. O'Connell Motherway M, Fitzgerald G F, Neirynck S, Ryan S, Steidler L, van Sinderen D. Characterization of ApuB, an extracellular type II amylopullulanase from Bifidobacterium breve UCC2003. Appl Environ Microbiol 2008; 74(20): 6271-6279.
56. Yasui H, Mike A, Ohwaki M. Immunogenicity of Bifidobacterium breve and change in antibody production in Peyer's patches after oral administration. J Dairy Sci 1989; 72(1): 30-35.
57. Moriyama E H, Niedre M J, Jarvi M T, Mocanu J D, Moriyama Y, Subarsky P et al. The influence of hypoxia on bioluminescence in luciferase-transfected gliosarcoma tumor cells in vitro. Photochem Photobiol Sci 2008; 7(6): 675-680.
58. Wiles S, Pickard K M, Peng K, MacDonald T T, Frankel G. In vivo bioluminescence imaging of the murine pathogen Citrobacter rodentium. Infect Immun 2006; 74(9): 5391-5396.
59. Cronin M, Akin A R, Collins S A, Meganck J, Kim J B, Baban C K, Joyce S A, van Dam G M, Zhang N, van Sinderen D, O'Sullivan G C, Kasahara N, Gahan C G, Francis K P, Tangney M. High resolution in vivo bioluminescent imaging for the study of bacterial tumour targeting. PLoS One 2012; 7: e30940.

What is claimed is:

1. A method of delivering an agent to an extra-intestinal site in the body, the method comprising:
orally administering a composition to the subject, the composition comprising at least one bacterial species adapted for oral administration, the bacterial species being one of linked to the agent and capable of expressing the agent,
wherein the bacterial species targets a site in the body and the agent is delivered one of within and adjacent to the site in the body.

2. The method of claim 1, wherein the site is one of an organ of the body, a tumour site, and the bloodstream.

3. The method of claim 1, wherein the bacterial species is a food grade, non-pathogenic, gram-positive bacteria capable of anaerobic growth.

4. The method of claim 3, wherein the food grade, non-pathogenic, gram-positive bacteria capable of anaerobic growth is an E. coli, Bifidobacteria, Lactobacillus or a Lactococcus species.

5. The method of claim 1, wherein the bacterial species is viable.

6. The method of claim 1, wherein the agent is encoded on a vector capable of expressing at least one gene, the vector being transformed into the bacterial species.

7. The method of claim 1, wherein the agent is linked to the bacterial species.

8. The method of claim 1, wherein the agent is selected from the group comprising:
an imaging agent, such as a fluorescent protein (e.g. RFP, GFP) a substrate-converting enzyme (e.g., Luciferase, bacterial lux operon, Alkaline Phosphatase, B-Galactosidase), or image contrast-enhancing agent, a tumour suppressing agent, such as an anti-angiogenic agent (e.g. endostatin, angiostatin, IL12, IL23, IL32), an immune cell regulating agent (cytokine or other), a secreted peptide, a tumour associated antigen, an antibody, a pro-apoptotic peptide (e.g. IL23), or a cell signalling interfering peptide (e.g. IL 32).

9. The method of claim 1, wherein the composition is adapted for use in at least one of a method of tumour detection, a method of tumour imaging, a method of monitoring the effectiveness of tumour treatment, a method of tumour staging, a method of detecting tumour metastasis, a method of tumour or cancer treatment, and a method of disease treatment or detection.

10. A method of one of tumour detection and tumour imaging, the method comprising:
(i) orally administering a composition to a subject, the composition comprising a bacterial species containing a vector capable of expressing at least one exogenous gene, wherein the bacterial species targets the tumour and the gene is expressed within or adjacent to the targeted tumour; and
(ii) at least one of external imaging, detecting, and measuring the expression of the gene, to allow one of detection and imaging of said tumours in the body.

11. The method of claim 10, wherein the method is adapted for monitoring the effectiveness of tumour treatment, tumour staging or detecting tumour metastasis.


SEQUENCE LISTING (8 sequences)

SEQ ID NO: 1 - DNA, 19 bp, Artificial Sequence
  Primer
ggtgtgaaagtccatcgct

SEQ ID NO: 2 - DNA, 20 bp, Artificial Sequence
  Primer
gtctgccaaggcatccacca

SEQ ID NO: 3 - DNA, 20 bp, Artificial Sequence
  Primer
gcgaggtaacctcgaacatg

SEQ ID NO: 4 - DNA, 20 bp, Artificial Sequence
  Primer
cggcgtatcgataattcacg

SEQ ID NO: 5 - DNA, 13 bp, Artificial Sequence
  Primer
ccggaatagctcc

SEQ ID NO: 6 - DNA, 20 bp, Artificial Sequence
  Primer
gaccatgcaccacctgtgaa

SEQ ID NO: 7 - DNA, 20 bp, Artificial Sequence
  Primer
ttccagttgatcgcatggtc

SEQ ID NO: 8 - DNA, 15 bp, Artificial Sequence
  Primer
cgtatctctacgacc